(19) United States
(12) Patent Application Publication
(10) Pub. No.: US 2007/0066970 A1
(43) Pub. Date: Mar. 22, 2007

(54) Integrated electrosurgical cart and surgical smoke evacuator unit

(76) Inventor: Leonard Ineson, Mississauga (CA)

Correspondence Address: LEONARD INESON, UNIT#33, 2280 MUNN'S AVENUE EAST, OAKVILLE, ON, L6H 3L1, CA

(21) Appl. No.: 11/227,153

(22) Filed: Sep. 16, 2005

(51) Int. Cl. A61B 18/04

(52) U.S. Cl. 606/34; 606/1

(57) ABSTRACT

An electrosurgical cart for use during surgery to support a surgical smoke evacuator unit comprises a first shelf for receiving and retaining a surgical smoke evacuator unit thereon. There is a substantially upright frame for structurally supporting the first shelf. There is also a plurality of wheels for rolling contact with a floor. An electrical control panel is mounted the electrosurgical cart, for controlling the surgical smoke evacuator unit. An electrical cable permits the transmission of electrical signals from the electrical control panel to the surgical smoke evacuator unit. A throughpassage on the front of the electrosurgical cart permits the flow of air from the inlet on the cart via a corrugated flexible hose to the filter on the smoke evacuation unit located on the bottom shelf.

### FIELD OF THE INVENTION

The present invention relates to carts for use in medical applications, such as surgery, and more particularly to such carts that are used to carry electrosurgical smoke evacuators.

### BACKGROUND OF THE INVENTION

During surgery, it is common for a surgeon to use an electrosurgical tool, commonly known as an electrosurgical pencil to cut tissue and coagulate blood vessels. The electrosurgical pencil includes an electrode tip that is operated by means of a cut-coagulate switch on the electrosurgical pencil, in order to perform tissue cutting and cauterization as required. A wire lead is connected at one end to the electrosurgical pencil. The other end of the wire lead plugs into a co-operating electrical socket on an electrosurgical unit (ESU), in order to supply power to the electrosurgical pencil. The electrosurgical unit itself has additional controls on it such as frequency, amplitude, suction strength, and so on, used by the nurse or surgeon to control the electrosurgical pencil.

During the cutting and cauterization processes, smoke is generated, which must be removed from the surgery site for various reasons. In order to draw surgical smoke away from the surgery site, the electrosurgical pencil is typically clipped onto a smoke evacuating tool that provides a suctioning function that draws the surgical smoke away from the surgery site. Alternatively, the electrosurgical pencil and smoke evacuating tool maybe integrated into a single device. The smoke evacuating tool that is attached to the electrosurgical pencil has an evacuation hose that plugs into a co-operating air inlet on a surgical smoke evacuator unit.

During surgery, the electrosurgical unit is typically placed on the top shelf of a wheeled cart that is located near the surgeon. The surgical smoke evacuator unit is typically placed on the bottom shelf of the wheeled cart. Accordingly, the the controls of the electrosurgical unit are difficult to reach during surgery, which is highly undesirable.

U.S. Pat. No. 5,518,310 issued May 21, 1996 to Ellman et al, discloses a Mobile Cart for Electrosurgical Instruments and Accessories Therefor. This mobile cart comprises a platform for supporting an electrosurgical instrument or the like, on the top shelf. An electrosurgical suctioning unit is disposed on the bottom shelf. Other configurations of supporting electrosurgical equipment are also possible. It can be readily seen that the electrosurgical smoke evacuator on the bottom shelf is extremely difficult to access during surgery, which is highly undesirable.

It is an object of the present invention to provide a cart for use during surgery to support a surgical smoke evacuator unit.

It is another object of the present invention to provide a cart for a medical smoke evacuator for use during surgery to support a surgical smoke evacuator unit, so as to be in easy reach for a surgeon during a surgical procedure.

### SUMMARY OF THE INVENTION

In accordance with one aspect of the present invention there is disclosed a novel electrosurgical cart for use during surgery to support a surgical smoke evacuator unit. The electrosurgical cart comprises a first shelf for receiving and retaining a surgical smoke evacuator unit thereon. There is a substantially upright frame for structurally supporting the first shelf. There is also a plurality of wheels for rolling contact with a floor. An electrical control panel is mounted the electrosurgical cart, for controlling the surgical smoke evacuator unit. There is also means for transmitting electrical signals from the electrical control panel to the surgical smoke evacuator unit.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described herein below.

### BRIEF DESCRIPTION OF THE DRAWINGS

The novel features which are believed to be characteristic of the electrosurgical cart according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIG. 1 is a perspective view from the front of the first preferred embodiment of the electrosurgical cart according to the present invention;

FIG. 2 is a perspective view from the rear of the first preferred embodiment electrosurgical cart of FIG. 1;

FIG. 3 is a side elevational view of the first preferred embodiment electrosurgical cart of FIG. 1;

FIG. 4 is a front end elevational view of the first preferred embodiment electrosurgical cart of FIG. 1;

FIG. 5 is a back end elevational view of the first preferred embodiment electrosurgical cart of FIG. 1;

FIG. 6 is a sectional top plan view of the first preferred embodiment electrosurgical cart of FIG. 1, taken along section line 6-6 of FIG. 3; and

FIG. 7 is an enlarged view of the control panel of the first preferred embodiment electrosurgical cart of FIG. 1; and,

FIG. 8 is an enlarged front end elevational view of the second preferred embodiment of the electrosurgical cart according to the present invention.

### DETAILED DESCRIPTION OF THE PREFERRED EMBODIMENTS

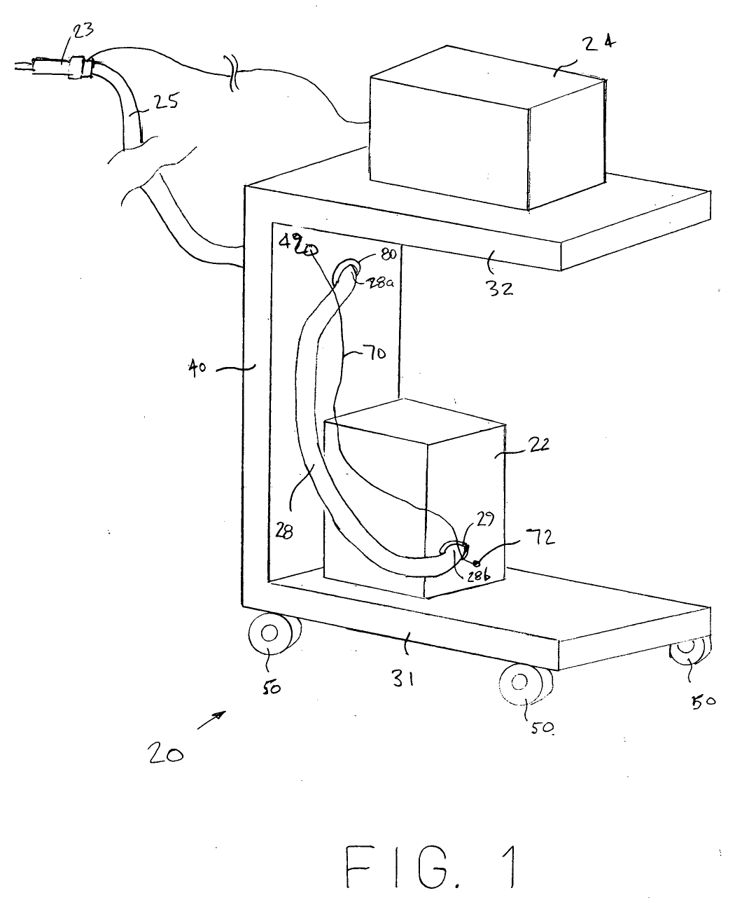

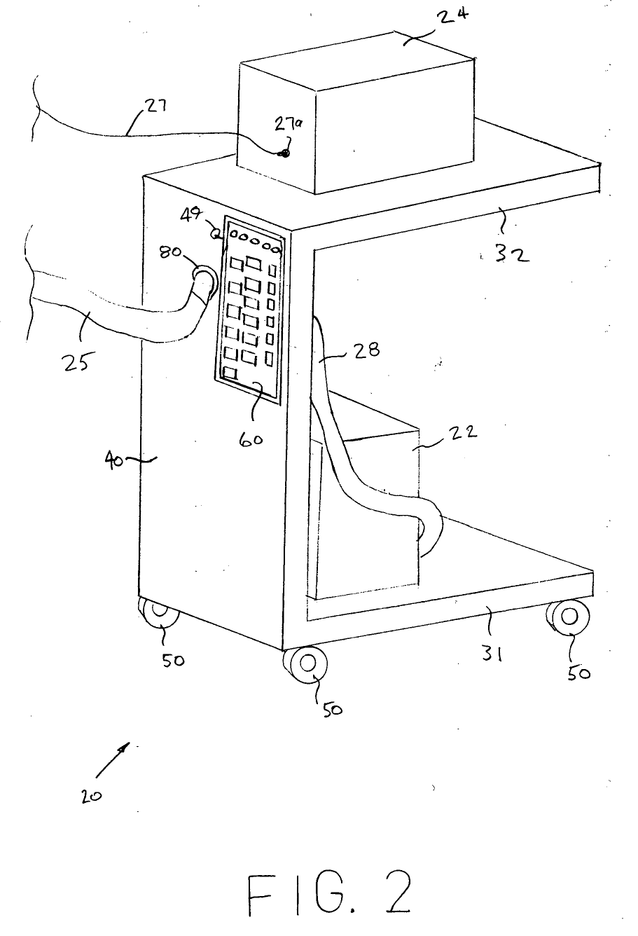

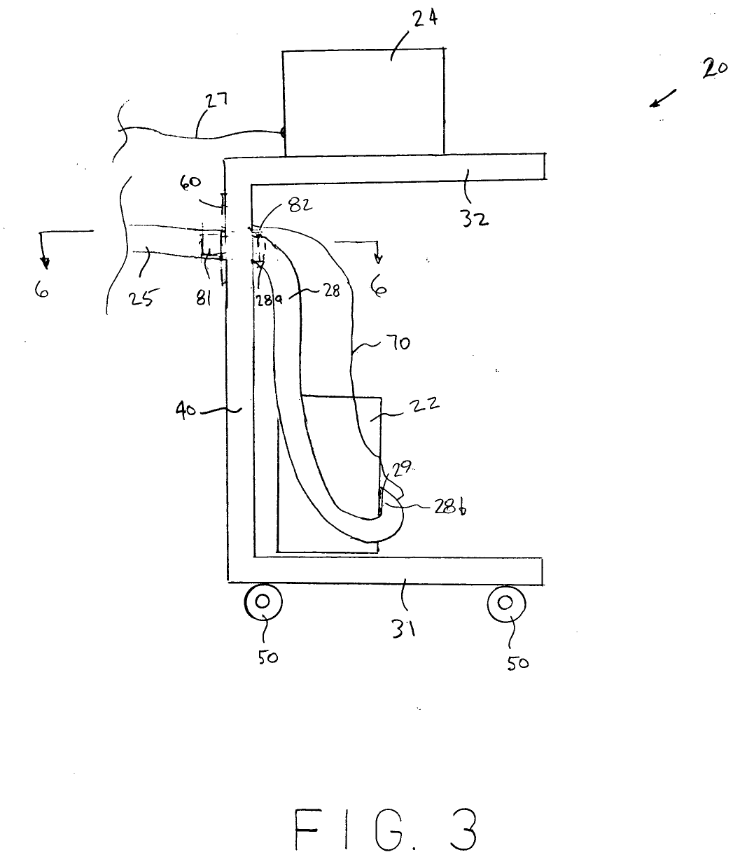

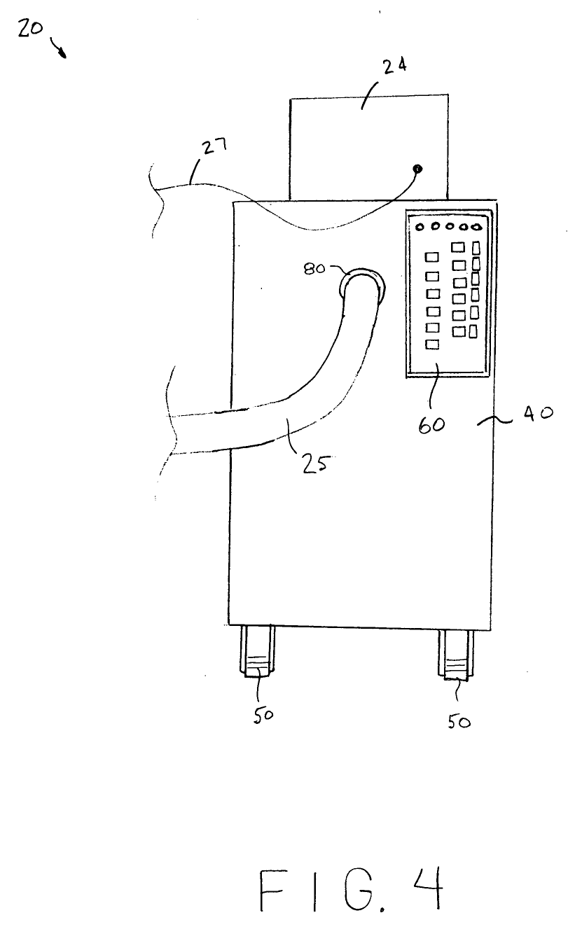

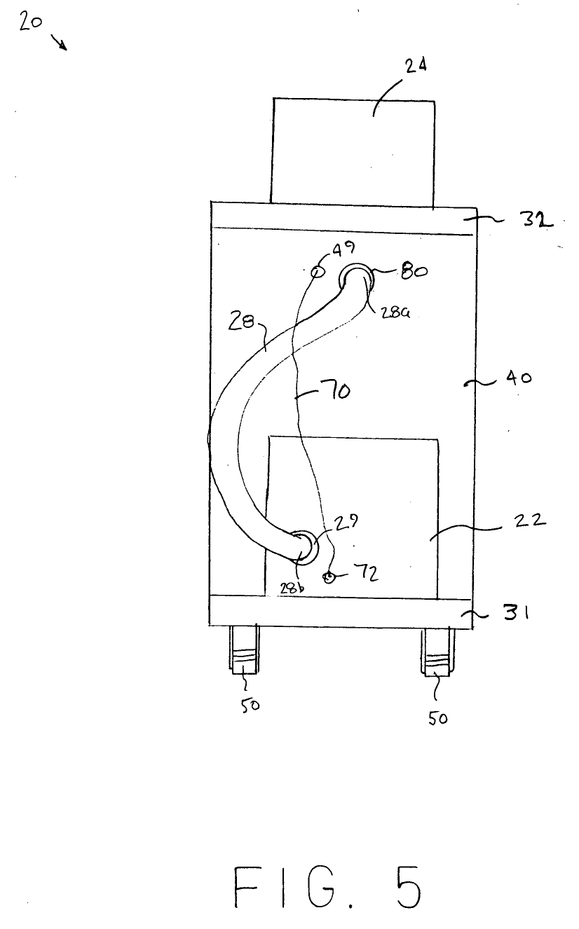

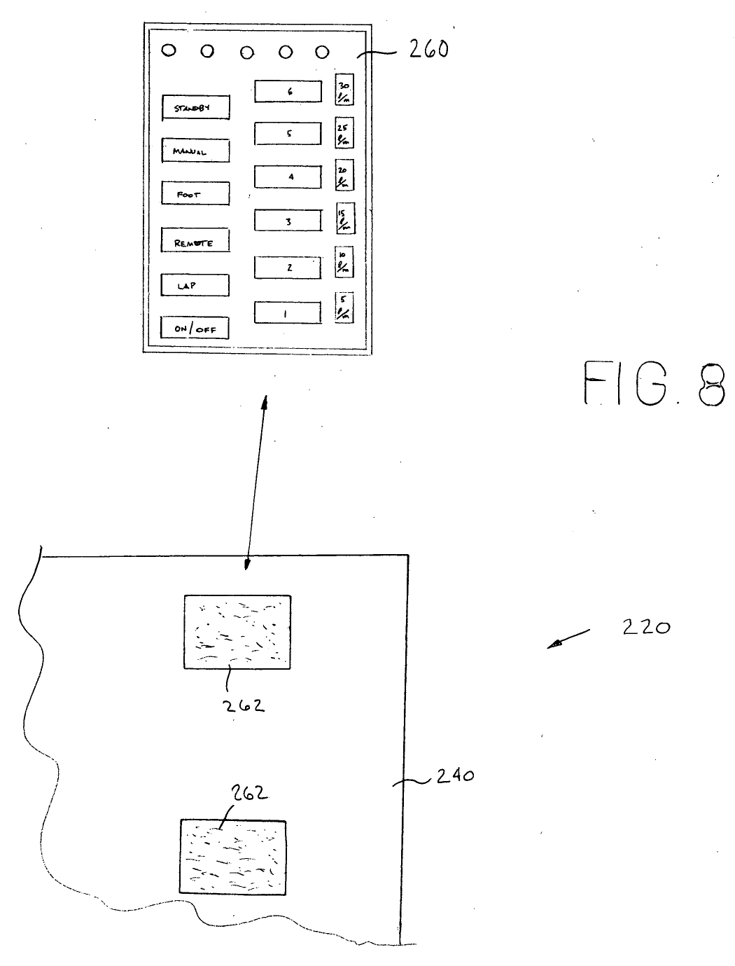

Referring to FIGS. 1 through 8 of the drawings, it will be noted that FIGS. 1 through 7 illustrate a first preferred embodiment of the electrosurgical cart of the present invention, and FIG. 8 illustrates a second preferred embodiment of the electrosurgical cart of the present invention.

Reference will now be made to FIGS. 1 through 7, which show a first preferred embodiment of the electrosurgical cart of the present invention, as indicated by general reference numeral 20. The electrosurgical cart 20 is for use during surgery to support a surgical smoke evacuator unit 22, and also typically support an electrosurgical unit 24.

The electrosurgical cart 20 comprises a first shelf 31 for receiving and retaining the surgical smoke evacuator unit 22 thereon. The surgical smoke evacuator unit 22 maybe any type of surgical smoke evacuator unit, and has an inlet 29 that conventionally receives the corrugated flexible hose 25 from a smoke evacuating tool 21 clipped to an electrosurgical pencil 23. With the electrosurgical cart 20 of the present invention, the flexible hose 25 from the smoke evacuating tool 21 is not connected to the inlet 29 of the surgical smoke evacuator unit 22, as will be discussed in greater detail subsequently. The power wire 27 of the electrosurgical pencil 23 is electrically connected to the appropriate socket on the electrosurgical unit 24, in a conventional manner.

The electrosurgical cart 20 further comprises a second shelf 32 for receiving and retaining medical equipment thereon, such as the electrosurgical unit 24, and/or any other necessary medical equipment. As can readily be seen in the figures, in the first preferred embodiment, the first shelf 31 is a bottom shelf and the second shelf 32 is a top shelf.

The electrosurgical cart 20 also comprises a substantially upright frame 40 for structurally supporting the first shelf 31 and the second shelf 32. In the first preferred embodiment as illustrated, the substantially upright frame 40 has a top end 42 and a bottom end 44, and forms the front of the electrosurgical cart 20 and is solid, with no openings. Alternatively, the substantially upright frame can have openings therein or could be more of a skeleton type frame, if desired.

A plurality of wheels 50 are mounted on the electrosurgical cart 20, specifically under the first shelf 31, for rolling contact with a floor. At least one of the wheels 50, and typically two of the wheels 50, have a locking means thereon to permit electrosurgical cart 20 to be selectively locked in place, as desired, to preclude it from rolling unwantedly.

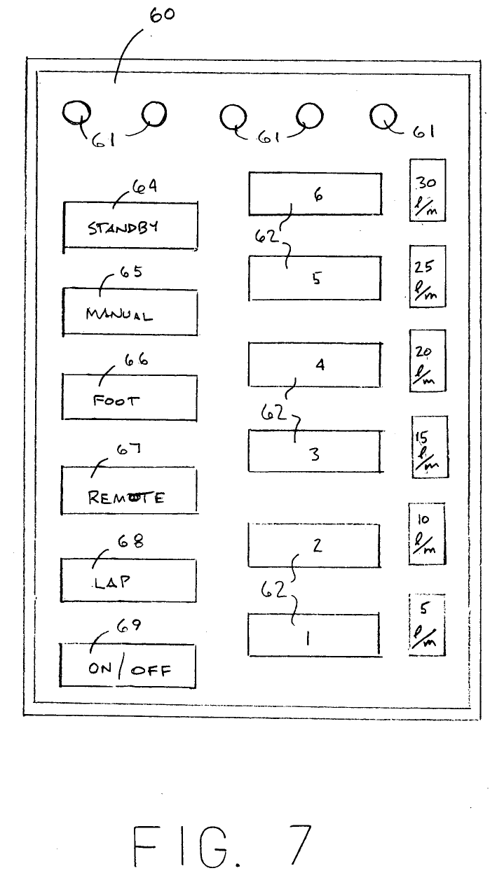

An electrical control panel 60 is mounted on the electrosurgical cart for controlling the surgical smoke evacuator unit 22. The electrical control panel 60 is preferably mounted on the substantially upright frame 40 so as to be readily accessible to a doctor or nurse using the electrosurgical cart 20. As can be best seen in FIG. 7, the electrical control panel 60 is a flat touch pad type control panel, so as to be substantially impervious to liquids, dirt, and so on. The electrical control panel 60 has a plurality of buttons thereon, such as speed select, buttons for the evacuating fan of the surgical smoke evacuator unit 22, and various function buttons. As shown, but not necessarily, there are six speeds ranging from five litres per minute to thirty litres per minute, with each speed being independently selectable by touching the appropriate button 62 on the electrical control panel 60. Further, button 64 permits selection of standby status of the surgical smoke evacuator unit 22, button 65 permits selection of the manual function of surgical smoke evacuator unit 22, button 66 permits selection of the foot control for the surgical smoke evacuator unit 22, button 67 permits selection of the remote control for the surgical smoke evacuator unit 22, button 68 permits selection of the lap function for surgical smoke evacuator unit 22, and button 68 permits the surgical smoke evacuator unit 22 be turned on and off. Other buttons with additional or alternative functions for the surgical smoke evacuator unit 22 may also be included on the control electrical control panel 60. Additionally, indicator lights 61 are included on the electrical control panel 60. These indicator lights indicate the status of the filter of the surgical smoke evacuator unit 22.

The electrosurgical cart 20 also comprises means for transmitting electrical signals from the control panel to the surgical smoke evacuator unit 22. In the first preferred embodiment as illustrated, the means for transmitting electrical signals from the electrical control panel 60 to the surgical smoke evacuator unit 22 comprises an electrical control cable 70 electrically connected interconnected between the electrical control panel 60 and the surgical smoke evacuator unit 22.

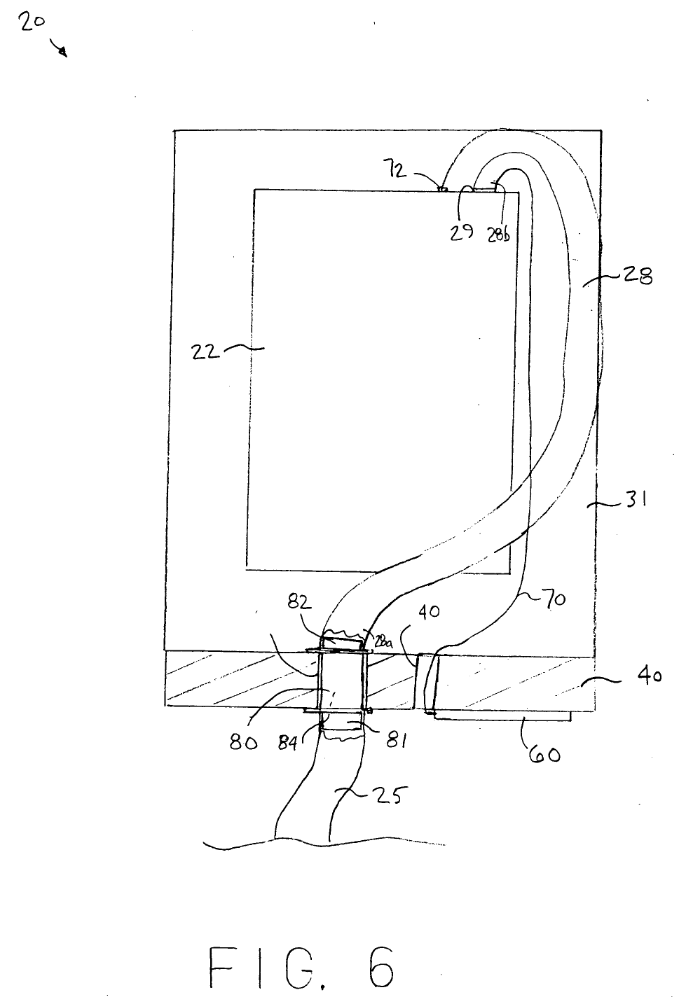

The electrosurgical cart 20 further comprises an aperture 49 in the substantially upright frame 40, as can be best seen in sectional FIG. 6, for receiving the electrical control cable 70 therethrough. The electrical control cable 70 extends from the from the electrical control panel 60 to a co-operating jack 72 on the surgical smoke evacuator unit 22.

A coupling member 80 is mounted on the electrosurgical cart 20, specifically in the substantially upright frame 40 at the aperture 49, so as to permit a passage through the substantially upright frame 40. The coupling member 80 has an inlet 81 for coupling the flexible hose 25 of the smoke evacuating tool 21 thereto. Alternatively, a pre-filter unit (not specifically shown) could be mounted on the inlet 81 of the coupling member 80, and the hose 25 of the smoke evacuating tool 21 could be coupled to the inlet of the pre-filter unit. The coupling member 80 also has an outlet 82 for coupling a secondary hose 28 thereto. The secondary hose 28 has a first end 28a coupled to the outlet 82 of the coupling member 80, and a second end 28b coupled to the inlet 29 of the surgical smoke evacuator unit 22. The coupling member 80 also comprises an internal throughpassage 84 for permitting the flow of air and surgical smoke therethrough. The coupling member 80 is preferably located adjacent the top end 42 of the substantially upright frame 40 so as to permit a person standing upright to readily couple the hose 25 of the smoke evacuating tool 21 to the inlet 81 of the coupling member 80 without bending over.

As can be readily seen in the figures, and is readily apparent from the description, the electrosurgical cart 20 permits ready control of a surgical smoke evacuator unit 22 disposed on the first shelf 31 through use of the electrical control panel 60, which is conveniently located for use by a doctor or nurse, during surgery.

Reference will now be made to FIG. 8, which shows a second preferred embodiment of the electrosurgical cart according to the present invention, as indicated by general reference numeral 220. The second alternative embodiment electrosurgical cart 220 is similar to the first preferred embodiment electrosurgical cart 20 except that the means for transmitting electrical signals from the electrical control panel 260 to the surgical smoke evacuator unit (not specifically shown) comprises a remote control unit 260. The remote control unit 260 is mounted on the substantially upright frame 240 of the electrosurgical cart 220 by means of a hook and loop fastener system 262, such as that sold under th trade name of Velcro™, or by any other suitable means. In this manner the remote control 260 can be used in place on the electrosurgical cart 220 or can be placed in any other convenient place at the surgical site, or can be held in the hand of a nurse or doctor.

As can be understood from the above description and from the accompanying drawings, the present invention provides an electrosurgical cart for use during surgery to support a surgical smoke evacuator unit, wherein the controls for the electrosurgical unit are on the cart, so as to be in easy reach for a surgeon during a surgical procedure, all of which features are unknown in the prior art.

Other variations of the above principles will be apparent to those who are knowledgeable in the field of the invention, and such variations are considered to be within the scope of the present invention. Further, other modifications and alterations may be used in the design and manufacture of the electrosurgical cart of the present invention without departing from the spirit and scope of the accompanying claims.

I claim:

1. An electrosurgical cart for use during surgery to support a surgical smoke evacuator unit, said electrosurgical cart comprising:
a first shelf for receiving and retaining a surgical smoke evacuator unit thereon;
a substantially upright frame for structurally supporting said first shelf;
a plurality of wheels for rolling contact with a floor;
an electrical control panel mounted said electrosurgical cart, for controlling said surgical smoke evacuator unit; and,
means for transmitting electrical signals from said electrical control panel to said surgical smoke evacuator unit.

2. The electrosurgical cart of claim 1, wherein said means for transmitting electrical signals from said control panel to said surgical smoke evacuator unit comprises an electrical control cable electrically interconnected between said electrical control panel and said surgical smoke evacuator unit.

3. The electrosurgical cart of claim 2, further comprising an aperture in said substantially upright frame for receiving said electrical control cable therethrough.

4. The electrosurgical cart of claim 3, further comprising a coupling member mounted on said cart at said aperture in said substantially upright frame, said coupling member having an inlet for coupling the hose of an electrosurgical pencil thereto, a throughpassage for permitting the flow of air and surgical smoke therethrough, and an outlet for coupling a secondary hose thereto, which secondary hose is also coupled to the inlet of said surgical smoke evacuator unit.

5. The electrosurgical cart of claim 4, further comprising a secondary hose having a first end coupled to said outlet said coupling member, and a second end coupled to the inlet a surgical smoke evacuator unit.

6. The electrosurgical cart of claim 1, further comprising a second shelf for receiving and retaining medical equipment thereon.

7. The electrosurgical cart of claim 6, wherein said first shelf is a bottom shelf and said second shelf is a top shelf.

8. The electrosurgical cart of claim 7, wherein said substantially upright frame structurally also supports said second shelf.

9. The electrosurgical cart of claim 1, wherein said electrical control panel is mounted on said substantially upright frame.

10. The electrosurgical cart of claim 1, wherein said means for transmitting electrical signals from said electrical control panel to said surgical smoke evacuator unit comprises a remote control unit.
